(19) United States
(12) Patent Application Publication
(10) Pub. No.: US 2026/0325764 A1
(43) Pub. Date: Oct. 22, 2026

(54) MULTI-NEEDLE ASSEMBLY FOR SKIN TREATMENT

(71) Applicants: Hyun Mee NOH, Yongin-si (KR); Si-Yeol BANG, Yongin-si (KR)

(72) Inventors: Hyun Mee NOH, Yongin-si (KR); Si-Yeol BANG, Yongin-si (KR)

(21) Appl. No.: 19/219,325

(22) Filed: May 27, 2025

(30) Foreign Application Priority Data
Apr. 18, 2025 (KR) 10-2025-0050646

(51) Int. Cl. A61M 5/32

(52) U.S. Cl. CPC A61M 5/3298; A61M 5/3202; A61M 2210/04

(57) ABSTRACT

Provided is a multi-needle assembly for skin treatment coupled to a discharge port of an injection device and including a guider including a distal end portion that contacts skin and includes a plurality of needle through-holes formed therein, a cap portion extending from the distal end portion and having a space inside, and arc-shaped fastening side walls symmetrically formed on both sides of an end of the cap portion and having a helical portion formed on an outer surface, an injection part inserted into the guider and coupled to the needle holder, in which the injection part includes, formed on one side, a withdrawal member including a plurality of injection solution holes formed therein, formed on the other side, an inlet pipe coupled with the discharge port of the injection device, and formed inside, a body including a formulation injection passage communicating with the inlet pipe and the withdrawal member, a needle holder coupled inside the guider, in which the needle holder includes a plurality of needles to pass through the needle through-holes, and is coupled to the withdrawal member of the injection part, an adjustment part including a threaded portion formed on an inner circumferential surface for screw-coupling with both fastening side walls of the guider, openings formed on both sides, and a toothed piece formed on the opening on one side for coupling with the injection part, in which the adjustment part raises and lowers the injection part so as to move the needle holder coupled to the withdrawal member and adjust a length of the needles.

### BACKGROUND

### Technical Field

The present disclosure relates to a multi-needle assembly for skin treatment, and more specifically, to a multi-needle assembly for skin treatment that enables accurate needle insertion into the skin without requiring a suction mechanism during a needle insertion process, and also allows for precise adjustment of a needle insertion depth in the skin with a simple operation.

### Description of Related Art

Skin treatments are performed to obtain rapid skin improvement effect by injecting formulation into the skin using multi-needles. The multi-needles are widely used to improve the convenience of the treatment by injecting formulation into large areas of skin, improve various skin problems such as wrinkles, spots, blemishes, stretch marks, acne, freckles, and pigmentation, maintain hair health by injecting formulation into the scalp, improve hair loss, and so on.

Korean Patent Application No. 10-2019-0003802, filed by the same inventor, proposes an “injection module for skin resurfacing”.

According to the above related art, the injection module includes: a main body coupled to a discharge port of a syringe; a needle pusher movable forward and backward along an inner circumferential surface of the main body, coupled with a plurality of needles, and provided with a connecting tube formed at a rear side to be connected to the discharge port of the syringe; and a suction adapter including a plurality of needle holes into which the plurality of needles are inserted, a contact surface formed on one side to contact the skin, and a suction port formed on the other side to be coupled with an opening of the main body so that a vacuum pressure is formed inside while the contact surface is in close contact with the skin. Therefore, by moving a cylindrical body coupled with the needles and screw-coupled to the suction adaptor, it is possible to rotate an adjustment unit and accurately adjust an exposed length of the needles for skin treatment.

However, the related multi-needle assembly for skin treatment has a problem in that the formulation leaks out and excessively spreads on the skin before the needles are removed, causing inconvenience to the patient.

In addition, there is a problem in that, before the needles are removed or before the needles are inserted into the skin, the formulation supplied to the outside from the needles flows into the suction adapter before being injected into the skin and leaks out through a gap at a coupling portion of the main body screw-coupled to the suction adapter.

In addition, the related multi-needle assembly for skin treatment requires a separate suction mechanism to accurately insert the needles into the skin during the process of inserting the needles into the skin, which leads to a complicated configuration and complex manufacturing and treatment procedure.

### SUMMARY

The present disclosure has been devised to improve the related invention, and it is an object of the present disclosure to provide a multi-needle assembly for skin treatment coupled to an injection device, which allows for injection treatment over a large area of subcutaneous tissue of the skin through a plurality of needles, can insert the needles into the skin accurately without requiring a suction mechanism, and can adjust the length of the needles in the process of inserting the needles into the skin so that the needles can be accurately inserted into the subcutaneous tissue, thereby improving the accuracy of the treatment.

According to an aspect, a multi-needle assembly for skin treatment coupled to a discharge port of an injection device is provided, which may include a guider including a distal end portion that contacts skin and includes a plurality of needle through-holes formed therein, a cap portion extending from the distal end portion and having a space inside, and arc-shaped fastening side walls symmetrically formed on both sides of an end of the cap portion and having a helical portion formed on an outer surface, an injection part inserted into the guider and coupled to the needle holder, in which the injection part includes, formed on one side, a withdrawal member including a plurality of injection solution holes formed therein, formed on the other side, an inlet pipe coupled with the discharge port of the injection device, and formed inside, a body including a formulation injection passage communicating with the inlet pipe and the withdrawal member, a needle holder coupled inside the guider, in which the needle holder includes a plurality of needles to pass through the needle through-holes, and is coupled to the withdrawal member of the injection part, an adjustment part including a threaded portion formed on an inner circumferential surface for screw-coupling with both fastening side walls of the guider, openings formed on both sides, and a toothed piece formed on the opening on one side for coupling with the injection part, in which the adjustment part raises and lowers the injection part so as to move the needle holder coupled to the withdrawal member and adjust a length of the needles.

The injection part may include an adjustment part coupling part formed on an outer circumferential surface of the body and coupled with the adjustment part.

The adjustment part coupling part may include an annular rib formed on the outer circumferential surface of the body of the injection part so as to be supported on an upper end of the fastening side wall of the guider, an annular plate formed on the outer circumferential surface of the body of the injection part while being spaced apart from the annular rib by a predetermined distance, and a fitting groove formed between the annular rib and the annular plate, into which the toothed piece of the adjustment part is fitted.

In a cross section, the toothed piece may include a downwardly inclined surface, and the annular plate may include an upwardly inclined surface formed on an outer circumferential surface, and the downwardly inclined surface is supported by and slides along the upwardly inclined surface.

The body of the injection part may protrude from an outer circumferential surface to form a protruding plate, the protruding plate may include first inclined surfaces formed on both side surfaces, the fastening side wall of the guider may include a stepped portion formed on an inner circumferential surface of an upper end, the stepped portion may include a second inclined surface formed on an outer surface, and the first inclined surface may pass along the second inclined surface and be coupled to the injection part and the guider.

The injection part may include an adhesive injection part formed to adhere the withdrawal member and the needle holder with an adhesive.

The injection part may include the adhesive injection part to adhere the withdrawal member and the needle holder with an adhesive, the adhesive injection part may includes an injection port formed in an annular plate of the injection part and an injection passage communicating with the injection port and formed inside the body, and the injection passage may be formed to communicate with the interior of the needle holder so that the adhesive is filled into an internal space of the needle holder.

The guider may include a bevel surface obliquely extending from the distal end portion, and the bevel surface may be inclined parallel to a needle inclined surface formed at an end of the needle.

The bevel surface may be formed with the same length as the width of the guider in the transverse direction, thereby defining a rectangular area.

The space of the cap portion of the guider may be formed so that an inner diameter thereof is decreased from the fastening side wall toward the distal end portion, the needle holder may be formed to be inclined downward to be in close contact with the inner wall surface of the space of the cap portion, an injection hole may be formed, and one end of the needle may be connected to the injection hole.

According to some aspects of the disclosure, there is an advantageous effect that the multi-needle assembly for skin treatment coupled to an injection device allows for injection treatment over a large area of subcutaneous tissue of the skin through a plurality of needles, and can insert the needles into the skin accurately without requiring a suction mechanism, and can adjust the length of the needles in the process of inserting the needles into the skin so that the needles can be accurately inserted into the subcutaneous tissue, thereby improving the accuracy of the treatment.

### BRIEF DESCRIPTION OF THE DRAWINGS

The above and other objects, features and advantages of the present disclosure will become more apparent to those of ordinary skill in the art by describing in detail exemplary aspects thereof with reference to the accompanying drawings, in which:

FIG. 1 is a perspective view of a multi-needle assembly for skin treatment according to an aspect of the disclosure;

FIG. 2 is a perspective view of the multi-needle assembly for skin treatment according to an aspect of the disclosure when viewed from the top;

FIG. 3 is a perspective view of the multi-needle assembly for skin treatment according to an aspect of the disclosure when viewed from the bottom;

FIG. 4 is an exploded perspective view of the multi-needle assembly for skin treatment according to an aspect of the disclosure;

FIG. 5 is a partial cross-sectional perspective view of the multi-needle assembly for skin treatment according to an aspect of the disclosure;

FIG. 6 is a front cross-sectional perspective view of the multi-needle assembly for skin treatment according to an aspect of the disclosure;

FIG. 7 is a front view showing the multi-needle assembly for skin treatment before an injection part is coupled to a guider according to an aspect of the disclosure; and

FIG. 8 is a front view showing the multi-needle assembly for skin treatment after the injection part is coupled to the guider according to an aspect of the disclosure.

### DETAILED DESCRIPTION

Hereinafter, preferred embodiments of the present disclosure will be described in detail with reference to the accompanying drawings.

The terms to be described below are defined in consideration of functions in the present disclosure, and it is noted that they should be interpreted as a concept consistent with the technical idea of the present disclosure and a meaning commonly or generally recognized in the art.

Further, in the following description of the present disclosure, a detailed description of known functions and configurations incorporated herein will be omitted when it may make the subject matter of the present disclosure rather unclear.

Here, the accompanying drawings are partially exaggerated or simplified for convenience and clarity of explanation and understanding of the configuration and operation of the technology, and each component does not exactly match the actual size.

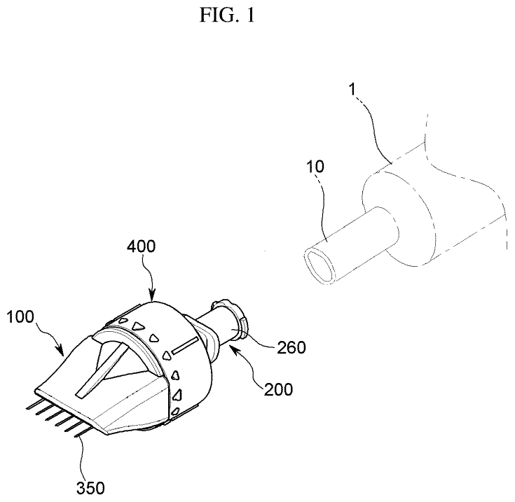

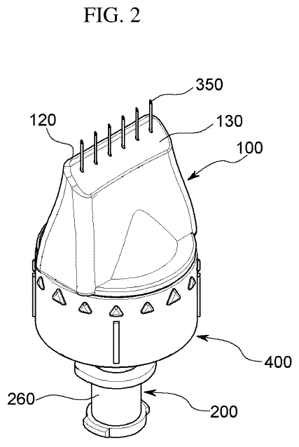

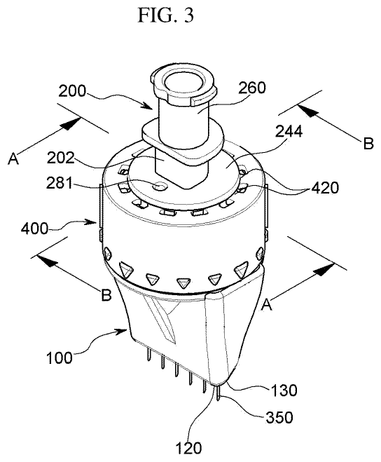

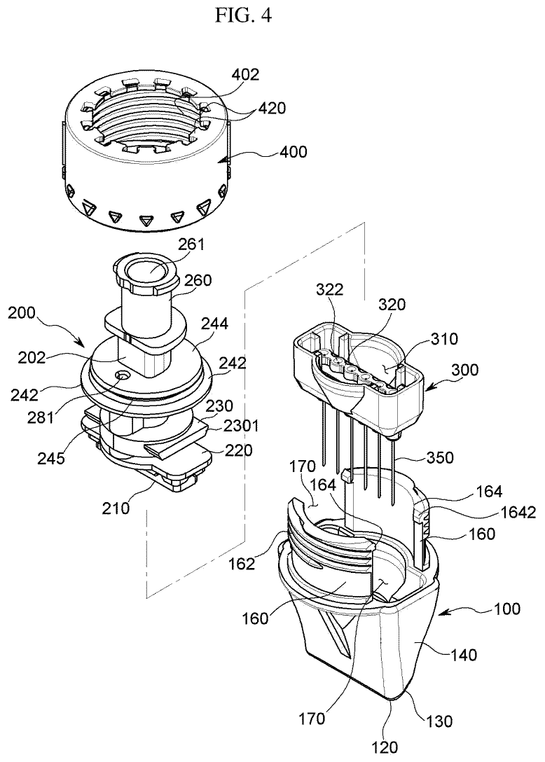

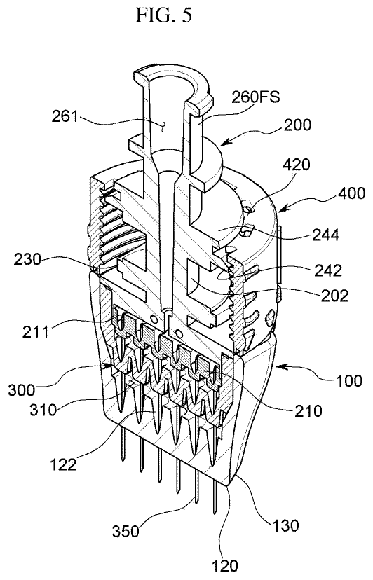

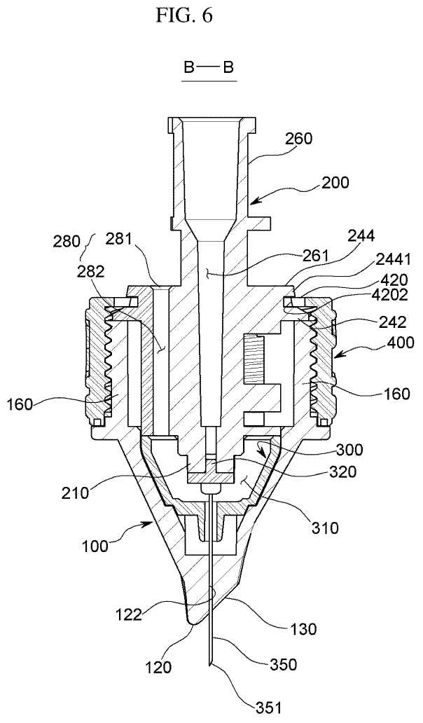

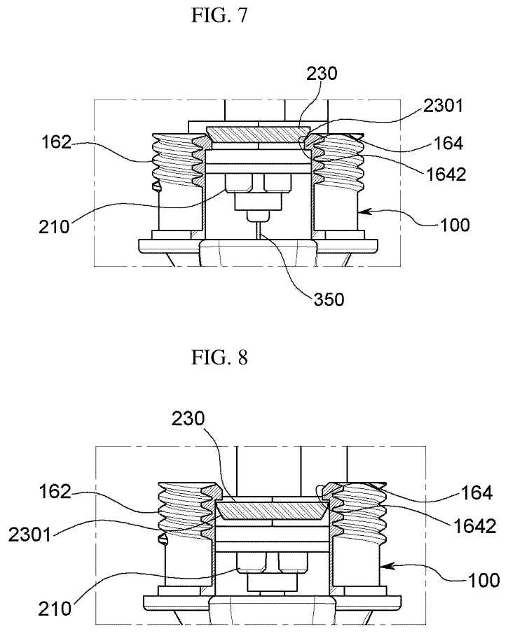

In the accompanying drawings, FIG. 1 is a perspective view of a multi-needle assembly for skin treatment according to an aspect of the disclosure, FIG. 2 is a perspective view of the multi-needle assembly for skin treatment according to an aspect of the disclosure when viewed from the top, FIG. 3 is a perspective view of the multi-needle assembly for skin treatment according to an aspect of the disclosure when viewed from the bottom, FIG. 4 is an exploded perspective view of the multi-needle assembly for skin treatment according to an aspect of the disclosure, FIG. 5 is a partial cross-sectional perspective view of the multi-needle assembly for skin treatment according to an aspect of the disclosure, FIG. 6 is a front cross-sectional perspective view of the multi-needle assembly for skin treatment according to an aspect of the disclosure, FIG. 7 is a front view showing the multi-needle assembly for skin treatment before an injection part is coupled to a guider according to an aspect of the disclosure, and FIG. 8 is a front view showing the multi-needle assembly for skin treatment after the injection part is coupled to the guider according to an aspect of the disclosure.

The multi-needle assembly for skin treatment according to an aspect of the disclosure is coupled to a discharge port 10 of an injection device 1, and mainly includes a guider 100, an injection part 200, a needle holder 300, and an adjustment part 400.

The guider 100 includes a distal end portion 120 that is brought into contact with the skin and that includes a plurality of needle through-holes 122, a cap portion 140 that extends from the distal end portion 120 and has a space inside, and arc-shaped fastening side walls 160 that are symmetrically formed on both sides of an end of the cap portion 140 and that include a helical portion 162 formed on an outer surface.

An insertion groove 170 is formed between the fastening side walls 160, which are symmetrically formed on both sides and spaced apart from each other. Through the insertion groove 170, a withdrawal member 210, a lower plate 220, and a protruding plate 230 of the injection part 200 may be inserted.

The injection part 200 is inserted into the guider 100 and coupled to the needle holder 300, and includes, formed on one side, the withdrawal member 210 with a plurality of injection solution holes 211 formed therein, formed on the other side, an inlet pipe 260 coupled with the discharge port 10 of the injection device 1, and, formed inside, a body 202 including a formulation injection passage 261 and connected to the inlet pipe 260 and the withdrawal member 210.

The withdrawal member 210 is formed below the body 202, and the lower plate 220 in a rectangular plate shape is formed above the withdrawal member 210.

The lower plate 220 may be coupled to an opening of the needle holder 300, sealing the interior of the needle holder 300.

The needle holder 300 is coupled inside the guider 100, includes a plurality of needles 350 passed through the needle through-holes 122 and coupled, and the opening formed at the top, and coupled so that the withdrawal member 210 of the injection part 200 is inserted into the injection solution holes 211 and the lower plate 220 blocks the opening.

An adhesive is injected and filled into the internal space of the needle holder 300 through an adhesive injection part 280 to be described below.

The adjustment part 400 includes a threaded portion 402 formed on an inner circumferential surface so as to be screw-coupled to both fastening side walls 160 of the guider 100, openings formed on both sides, and a toothed piece 420 formed on the opening on one side to be coupled with the injection part 200.

By rotating the adjustment part 400 in a screw-like manner, the injection part 200 is raised and lowered, thereby raising or lowering the needle holder 300 coupled to the withdrawal member 210 to adjust the length of the needles 350. That is, when the needles 350 pass through and are exposed from the distal end portion of the guider 100, the length by which the needles are exposed may be adjusted. The adjustment part 400 may include marks such as numbers, scales, and shapes formed on the surface, which indicate the treatment depth and allow for easy adjustment of the exposure length of the needles 350. In addition, the adjustment part 400 may include protrusions formed on the surface to prevent hand from slipping in the process of rotating the adjustment part 400 by hand.

Meanwhile, the injection part 200 includes an adjustment part coupling part 240 formed on an outer circumferential surface of the body 202 to be coupled with the adjustment part 400.

The adjustment part coupling part 240 includes an annular rib 242 formed on the outer circumferential surface of the body 202 of the injection part 200 so as to be supported on an upper end of the fastening side wall 160 of the guider 100, an annular plate 244 formed on the outer circumferential surface of the body 202 of the injection part 200 while being spaced apart from the annular rib 242 by a predetermined distance, and a fitting groove 245 formed between the annular rib 242 and the annular plate 244, into which the toothed piece 420 of the adjustment part 400 is fitted.

The toothed piece 420 of the adjustment part 400 is integrally coupled to the fitting groove 245 between the annular rib 242 and the annular plate 244. In an aspect, in cross-section, the toothed piece 420 includes a downwardly inclined surface 4202 and the annular plate 244 includes an upwardly inclined surface 2441 formed on an outer circumferential surface, so that the downwardly inclined surface 4202 of the toothed piece 420 is supported by and slides along the upwardly inclined surface 2441, making it easy for the adjustment part 400 to pass through the annular plate 244 of the injection part 200, and the toothed piece 420 is coupled to the fitting groove 245.

The body 202 of the injection part 200 protrudes from the outer circumferential surface to form the protruding plate 230. The fastening side wall 160 of the guider 100 includes a stepped portion 164 formed on an inner circumferential surface of an upper end thereof.

First inclined surfaces 2301 are formed on both side surfaces of the protruding plate 230 protruding from the outer circumferential surface of the body 202 of the injection part 200, and second inclined surfaces 1642 are formed on the outer surfaces of the stepped portions 164 formed on the inner circumferential surfaces of the upper ends of the fastening side walls 160 of the guider 100. Therefore, during the process of passing the injection part 200 between both fastening side walls 160 of the guider 100 and coupling the same, the first inclined surface 2301 of the protruding plate 230 can easily pass along the second inclined surface 1642 of the stepped portion 164, thereby facilitating coupling.

In addition, once the body 202 of the injection part 200 is coupled with the inner sides of both fastening side walls 160 of the guider 100, the injection part 200 is not separated from the guider 100, thereby preventing disengagement. Since the injection part 200 is moved inside the fastening side walls 160, and the plurality of needles 350 of the needle holder 300 are moved together according to the movement of the injection part 200, the protrusion length of the plurality of needles 350 extending through the needle through-holes 122 of the guider 100 may be adjusted, and by adjusting the protrusion length of the plurality of needles 350, the treatment depth of the plurality of needles 350 injected into the skin may be adjusted.

The injection part 200 includes the adhesive injection part 280 formed to adhere the withdrawal member 210 and the needle holder 300 of the injection part 200 with an adhesive.

Referring to FIG. 6, the adhesive injection part 280 includes an injection port 281 formed in the annular plate 244 of the injection part 200, and an injection passage 282 formed inside the body 202 and communicating with the injection port 281, in which the injection passage 282 is formed to communicate with the interior of the needle holder 300 so that the adhesive is filled in the internal space of the needle holder 300.

The guider 100 includes a bevel surface 130 obliquely extending from the distal end portion 120.

The bevel surface 130 is formed so that it is inclined at the same angle as a needle inclined surface 351, which is formed at an end of the needle 350, so as to be parallel to the needle inclined surface 351. Since the needle inclined surface 351 and the bevel surface 130 are formed to be inclined at the same angle, pain during the injection treatment may be reduced.

In addition, the bevel surface 130 is formed to have the same length as the width of the guider 100 in the transverse direction, thereby defining a rectangular area and reducing pain. That is, since the needle inclined surface and the bevel surface are formed at the same angle, the plurality of needles may be formed with the same needle length, and since formulation is injected along the needle inclined surface, the needles may be uniformly inserted into the skin, thereby reducing pain, effectively delivering formulation, and minimizing formulation loss.

Meanwhile, referring to FIGS. 4 and 6, the space of the cap portion 140 of the guider 100 is formed so that its inner diameter is decreased from the fastening side wall 160 toward the distal end portion 120.

In addition, the needle holder 300 is inclined downward so as to be in close contact with the inner wall surface of the space of the cap portion 140, and includes a protruding end portion 320 with an injection hole 322 formed therein, in which one end of the needle 350 is passed through the injection hole 322 and coupled, and the one end of the needle 350 is connected to the withdrawal member 210 of the injection part 200.

The protruding end portion 320 with the injection hole 322 formed therein is coupled to the fitting groove 245 formed in the withdrawal member 210 of the injection part 200.

One end of the needle 350 is coupled to the injection solution hole 211 of the withdrawal member 210 of the injection part 200. Therefore, the formulation injected from the inlet pipe 260 of the injection part 200 may be supplied to the needle 350 through the injection solution hole 211 of the withdrawal member 210.

The operation of the present disclosure configured as described above will be described below.

The needle holder 300 is coupled inside the guider 100 so that the needles 350 are exposed through the distal end portion thereof.

The lower plate 220 and the protruding plate 230 are inserted into the insertion groove 170 between both fastening side walls 160 of the guider 100 and the injection part 200 is coupled to the guider 100.

The adjustment part 400 is screw-coupled to the helical portion 162 of both fastening side walls 160 of the guider 100, and the toothed piece 420 is fitted into the fitting groove 245 between the annular rib 242 and the annular plate 244 so that they are integrated as one body. Therefore, by rotating the adjustment part 400, the injection part 200 is moved in the front-rear direction, and the needle holder 300 connected thereto is moved back and forth, allowing for the adjustment of the exposure length of the needles 350.

The present disclosure is not limited by the embodiments described above and the accompanying drawings, and various modifications and applications not illustrated within the scope not departing from the technical spirit of the present disclosure are possible, as well as substitution of components and change to other equivalent embodiments are possible, and accordingly, contents related to modifications and applications of the features of the present disclosure should be construed to be included in a scope of the present disclosure.

1. A multi-needle assembly for skin treatment coupled to a discharge port of an injection device, comprising:
a guider including a distal end portion that contacts skin and includes a plurality of needle through-holes formed therein, a cap portion extending from the distal end portion and having a space inside, and fastening side walls symmetrically formed on both sides of an end of the cap portion and having a helical portion formed on an outer surface;
an injection part inserted into the guider and coupled to the needle holder, wherein the injection part includes, formed on one side, a withdrawal member including a plurality of injection solution holes formed therein, formed on the other side, an inlet pipe coupled with the discharge port of the injection device, and formed inside, a body including a formulation injection passage communicating with the inlet pipe and the withdrawal member;
a needle holder coupled inside the guider, wherein the needle holder includes a plurality of needles to be passed through the needle through-holes and coupled, and is coupled to the withdrawal member of the injection part;
an adjustment part including a threaded portion formed on an inner circumferential surface for screw-coupling with both fastening side walls of the guider, openings formed on both sides, and a toothed piece formed on the opening on one side for coupling with the injection part, wherein the adjustment part raises and lowers the injection part so as to move the needle holder coupled to the withdrawal member and adjust a length of the needles.

2. The multi-needle assembly for skin treatment according to claim 1, wherein
the injection part includes an adjustment part coupling part formed on an outer circumferential surface of the body and coupled with the adjustment part, and
the adjustment part coupling part includes:
an annular rib formed on the outer circumferential surface of the body of the injection part so as to be supported on an upper end of the fastening side wall of the guider;
an annular plate formed on the outer circumferential surface of the body of the injection part while being spaced apart from the annular rib by a predetermined distance; and
a fitting groove formed between the annular rib and the annular plate, into which the toothed piece of the adjustment part is fitted.

3. The multi-needle assembly for skin treatment according to claim 2, wherein,
in a cross section, the toothed piece includes a downwardly inclined surface, and the annular plate includes an upwardly inclined surface formed on an outer circumferential surface, and
the downwardly inclined surface is supported by and slides along the upwardly inclined surface.

4. The multi-needle assembly for skin treatment according to claim 1, wherein
the body of the injection part protrudes from an outer circumferential surface to form a protruding plate,
the protruding plate includes first inclined surfaces formed on both side surfaces,
the fastening side wall of the guider includes a stepped portion formed on an inner circumferential surface of an upper end,
the stepped portion includes a second inclined surface formed on an outer surface, and
the first inclined surface passes along the second inclined surface and is coupled to the injection part and the guider.

5. The multi-needle assembly for skin treatment according to claim 1, wherein
the injection part includes an adhesive injection part formed to adhere the withdrawal member and the needle holder with an adhesive,
the adhesive injection part includes an injection port formed in an annular plate of the injection part and an injection passage communicating with the injection port and formed inside the body, and
the injection passage is formed to communicate with the interior of the needle holder so that the adhesive is filled into an internal space of the needle holder.

6. The multi-needle assembly for skin treatment according to claim 1, wherein
the guider includes a bevel surface obliquely extending from the distal end portion, and
the bevel surface is inclined parallel to a needle inclined surface formed at an end of the needle.

7. The multi-needle assembly for skin treatment according to claim 1, wherein
the needle holder is formed to be inclined downward to be in close contact with an inner wall surface of the space of the cap portion,
the space of the guider cap portion is formed so that an inner diameter thereof is decreased from the fastening side walls toward the distal end portion,
the needle holder includes a protruding end portion with an injection hole formed inside, and
one end of the needle is passed through the injection hole and coupled.
